(19) United States
(12) Patent Application Publication
(10) Pub. No.: US 2015/0190749 A1
(43) Pub. Date: Jul. 9, 2015

(54) Method and Apparatus for Estimating the Amount of Reductant Slip in a Selective Catalytic Reduction Device

(71) Applicant: Perkins Engines Company Limited, Peterborough, Cambridgeshire (GB)

(72) Inventors: Gavin Williams, Stamford (GB); Matthew Nicholson, Peterborough (GB); Stuart Smith, Stamford (GB)

(73) Assignee: Perkins Engines Company Limited, Peterborough, Cambridgeshire (GB)

(21) Appl. No.: 14/407,703

(22) PCT Filed: Jun. 26, 2013
(86) PCT No.: PCT/GB2013/051695

§ 371 Date: Dec. 12, 2014

(30) Foreign Application Priority Data
Jun. 26, 2012 (GB) 1211293.4

(51) Int. Cl. B01D 53/34; B01D 53/56; B01D 53/86; G01N 33/00

(52) U.S. Cl. CPC B01D 53/346; G01N 33/0004; B01D 53/56; B01D 53/8625; B01D 2257/404; B01D 2251/2062

(57) ABSTRACT

The present disclosure provides a method and apparatus for estimating the amount of ammonia output from an SCR device by determining a NOx conversion efficiency of the SCR device using a variance of a NOx input measurement and a variance of a NOx output measurement, obtaining an estimate of NOx output from the SCR device using the NOx conversion efficiency and using that estimate to determine an estimate of ammonia output from the SCR device.

### TECHNICAL FIELD

This disclosure relates to methods and apparatuses for selective catalytic reduction (SCR) device slip detection.

### BACKGROUND

Selective catalytic reduction (SCR) devices may be used to convert nitrous oxides (NOx), which may be produced, for example, by an internal combustion engine, into less harmful emissions, such as nitrogen and water. The SCR device may comprise a catalyst that facilitates a reaction between the NOx, which may be present in a gas stream passing through the SCR device, and a reductant in order substantially to remove the NOx from the gas stream.

The reductant may be added to the gas stream and absorbed onto the catalyst before it reacts with the NOx in the gas stream passing through the SCR device. Where the reductant used is ammonia, it may be added to the gas stream as, for example, anhydrous ammonia, aqueous ammonia or urea that thermally decomposes into ammonia within the SCR device before being absorbed onto the catalyst.

When the SCR device is dosed with reductant correctly, the reaction between the ammonia and NOx should eliminate nearly all of the NOx and ammonia. If the SCR device is over dosed, there may be more ammonia within the SCR device than can be absorbed onto the catalyst, which may result in ammonia being emitted from the SCR device (commonly known as ‘ammonia slip’). Ammonia emissions are undesirable as they can be very harmful to the environment. If the system is under-dosed, there may be insufficient ammonia absorbed onto the catalyst to react with all of the NOx passing through the SCR device, which may result in unprocessed NOx being emitted from the SCR device. This may reduce the conversion efficiency of the SCR device and is therefore also undesirable.

In order to control the level of dosing more accurately, it may be desirable to monitor the amount of NOx and ammonia slip at the output of the SCR device. However, NOx sensors are cross sensitive to ammonia. Consequently, a high reading from the NOx out sensor may be the result of either untreated NOx being emitted, which is caused by under-dosing, or by ammonia slip, which is caused by over-dosing.

Therefore, it may be difficult to tell from the reading of a NOx sensor at the output of the SCR device whether the SCR device is being under-dosed or-over dosed. This makes effective dosing control more difficult.

It may be possible to use an ammonia sensor in conjunction with a NOx sensor at the output of the SCR device in order to differentiate between NOx emissions and ammonia slip. However, an ammonia sensor is an expensive additional component. Therefore, it may be undesirable to use an ammonia sensor at the output of an SCR device.

International patent application no. WO2006000877 suggests a control technique intended to overcome the problems of NOx sensor cross-sensitivity. In the method suggested by the application, a pulse may be introduced in the ammonia feed rate into an SCR device. The change in reading from a NOx sensor downstream of the SCR device is monitored. An increased reading in response to a positive pulse indicates that ammonia slip is occurring and that dosing levels should therefore be decreased. A decreased reading in response to a positive pulse indicates that untreated NOx is being emitted from the SCR device and that dosing levels should therefore be increased.

However, this technique relies on interrupting the normal dosing regime of the SCR device in order to introduce the pulse and then monitor its effect. Not only does this require time, both to allow the pulse of ammonia dosing to have an effect on the output of the device and for computation, but often the SCR device will be tested whilst already overdosing and the pulse in dosing may increase the ammonia slip even further for a period of time. In the converse case of under-dosing, conversion efficiency will be further reduced. Furthermore, it only indicates whether or not over-dosing is occurring and cannot give an accurate indication of the extent of over-dosing, which might be useful for subsequent dosing control. In addition, at very large catalyst storage release, ammonia slip is predominantly derived from the catalyst itself, and very little from the dosing means. The correlation between dosing and the NOx out reading from the downstream NOx sensor is lost, which can result in loss of detection of ammonia.

### SUMMARY

The disclosure provides: a method for estimating the amount of ammonia output from a selective catalytic reduction (SCR) device, the method comprising the steps of: determining the NOx conversion efficiency of the SCR device from the variance of a NOx sensor measurement signal at the input of the SCR device and the variance of a NOx sensor measurement signal at the output of the SCR device; estimating the amount of NOx output from the SCR device from the NOx conversion efficiency of the SCR device and the NOx sensor measurement signal at the input to the SCR device; and estimating the amount of ammonia output from the SCR device from the estimated NOx output and the NOx sensor measurement signal at the output of the SCR device.

The disclosure also provides: a controller for estimating the amount of ammonia output from a selective catalytic reduction (SCR) device, the controller being configure to: determine the NOx conversion efficiency of the SCR device from the variance of a NOx sensor measurement signal at the input of the SCR device and the variance of a NOx sensor measurement signal at the output of the SCR device; estimate the amount of NOx output from the SCR device from the NOx conversion efficiency of the SCR device and the NOx sensor measurement signal at the input to the SCR device; and estimate the amount of ammonia output from the SCR device from the estimated NOx output and the NOx sensor measurement signal at the output of the SCR device.

### BRIEF DESCRIPTION OF THE DRAWINGS

FIG. 1 shows a schematic drawing of an engine unit comprising an SCR device;

FIG. 2 shows the method steps that may be used to estimate the ammonia output from the SCR device of FIG. 1;

FIG. 3 shows the steps that may be undertaken to determine if the SCR device of FIG. 1 should continue to be dosed or if dosing should cease; and

FIG. 4 shows an example vehicle within which the engine unit shown in FIG. 1 may be used.

### DETAILED DESCRIPTION

An SCR device may be used in a variety of different applications where a reduction in NOx levels in a gas stream is desired. Such applications may include, but are not limited to, boilers, gas turbines and internal combustion engines, for example diesel engines.

FIG. 1 shows an internal combustion engine 10 with an SCR device 20 at the exhaust of the internal combustion engine 10.The SCR device 20 in this arrangement may be dosed by injecting urea into the exhaust gas upstream of the SCR device 20 with an injector 40, although any other suitable dosing agent, for example anhydrous or aqueous ammonia, may alternatively be used and added to the SCR device 20 using any suitable technique known to the skilled person.

A first (or upstream) NOx sensor 42 may be located upstream of the SCR device 20 in order to generate a signal indicating the amount of NOx input (NOxᵢₙ) to the SCR device 20. A second (or downstream) NOx sensor 44 may be located downstream of the SCR device 20 in order to generate a signal indicating the amount of NOx output (NOxₒᵤₜ) from the SCR device 20.

In order to control the dosing of the SCR device 20, a ‘cut dosing flag’ control signal may be used. If it is determined that ammonia slip may be taking place, the ‘cut dosing control flag’ may be set high and dosing of the SCR device 20 be suspended. If it is determined that ammonia slip may not be taking place, the ‘cut dosing flag’ may be set low and dosing of the SCR device 20 be re-commenced and allowed to continue until the ‘cut dosing flag’0 is set high again.

There are many different ways in which the state of the ‘cut dosing flag’ may be determined. These shall be explained below.

NOx sensors are cross sensitive to ammonia. This cross sensitivity should not affect the upstream NOx sensor 42 since it is located upstream of the SCR device 20 and the injector 40 and may not come into contact with ammonia. Consequently, the measurement signal NOxᵢₙ should give an accurate indication of the amount of NOx in the exhaust gas upstream of the SCR device 20.

However, the downstream NOx sensor 44 may be affected by cross-sensitivity with ammonia. If the SCR device 20 is overdosed with urea, ammonia slip may occur. When this happens, the NOxₒᵤₜ signal from the downstream NOx sensor 44 may be made up of two components: the amount of NOx output from the SCR device 20 and the amount of ammonia output from the SCR device 20. Thus, the NOxₒᵤₜ signal from the downstream NOx sensor 44 may not give an accurate indication of the amount of NOx in the exhaust gas downstream of the SCR device 20.

If NOxₒᵤₜ very is low, for example below 20 ppm or below 10 ppm, it may not matter whether NOx or ammonia is being output from the SCR device 20 because it is being output in a low enough quantity not to be important. However, if NOxₒᵤₜ is higher, it may be important to investigate if it is NOx or ammonia that is being output from the SCR device 20, so that dosing may be adjusted accordingly.

It may be possible to differentiate between NOx output from the SCR device 20 and ammonia output from the SCR device by considering the NOx conversion efficiency (ε) of the SCR device 20.

The conversion efficiency (ε) may be obtained either from the mean of NOxᵢₙ and the mean of NOxₒᵤₜ, or from the variance of NOxᵢₙ and the variance of NOxₒᵤₜ.

The conversion efficiency based on mean values (the average NOx conversion efficiency, εₘₑₐₙ) may be calculated as follows:

$${ɛ}_{mean} = \frac{\overline{{NOx}_{i n}} - \overline{{NOx}_{out}}}{\overline{{NOx}_{i n}}}$$

The conversion efficiency based on variance values (_{εvar}) may be calculated as follows:

$${ɛ}_{var} = \frac{var ( {NOx}_{i n} ) - var ( {NOx}_{out} )}{var ( {NOx}_{i n} )}$$

It may be immediately appreciated that rather than using the variance of NOxᵢₙ and NOxₒᵤₜ, the standard deviation of the signal may be used, since standard deviation is the square root of variance. Consequently, analogous results for efficiency may also be obtained by considering standard deviation. However, for the sake of clarity, efficiency based on standard deviation shall not be discussed herein any further.

In both mean and variance measurements of efficiency, when the SCR device 20 is working efficiently and NOxₒᵤₜ is zero, ε will be equal to 1. When the SCR device 20 is working inefficiently and NOxₒᵤₜ is equal to NOxᵢₙ, will be equal to 0.

When only NOx is passing out of the SCR device 20, the NOxₒᵤₜ measurement from the downstream NOx sensor 44 should give an accurate indication of the amount of NOx in the exhaust gas output from the SCR device 20 and εₘₑₐₙ should be similar to ε_{var.}

However, when ammonia slip is taking place, the NOxₒᵤₜ measurement from the downstream NOx sensor 44 may comprise two components: the amount of NOx output from the SCR device 20 and the amount of ammonia output from the SCR device 20. Thus, mean NOxₒᵤₜ may be greater than the actual mean amount of NOx output from the SCR device 20. This will result in a lower εₘₑₐₙ than the actual efficiency of the SCR device 20.

In contrast, εᵥₐᵣ should continue to reflect the actual conversion efficiency of the SCR device 20 more accurately. This is because during ammonia slip, var(NOxₒᵤₜ) comprises a higher-frequency NOx output variance component that is superposed on top of a lower frequency ammonia output variance component. Since low frequency variations caused by the ammonia content within the NOxₒᵤₜ signal may have very little or no effect on the variance of NOxₒᵤₜ, εᵥₐᵣ may not be affected to any significant degree by ammonia slip.

In consequence, when ammonia slip is taking place, εₘₑₐₙ may no longer be similar to εᵥₐᵣ Instead, εₘₑₐₙ may be less than ε_{var.}

Because εᵥₐᵣ may provide an accurate indication of the conversion efficiency of the SCR device 20, even during ammonia slip, it may be used to calculate an estimate of the actual NOx output (est(NOxₒᵤₜ)) and ammonia output (est(NH₃)) from the SCR device 20. The estimate of NOx output may be more accurate than the NOxₒᵤₜ measurement obtained from the downstream NOx sensor 24, which may be affected by ammonia cross-sensitivity. By obtaining a more accurate estimate of NOx output, a more accurate estimate of ammonia output may also be obtained.

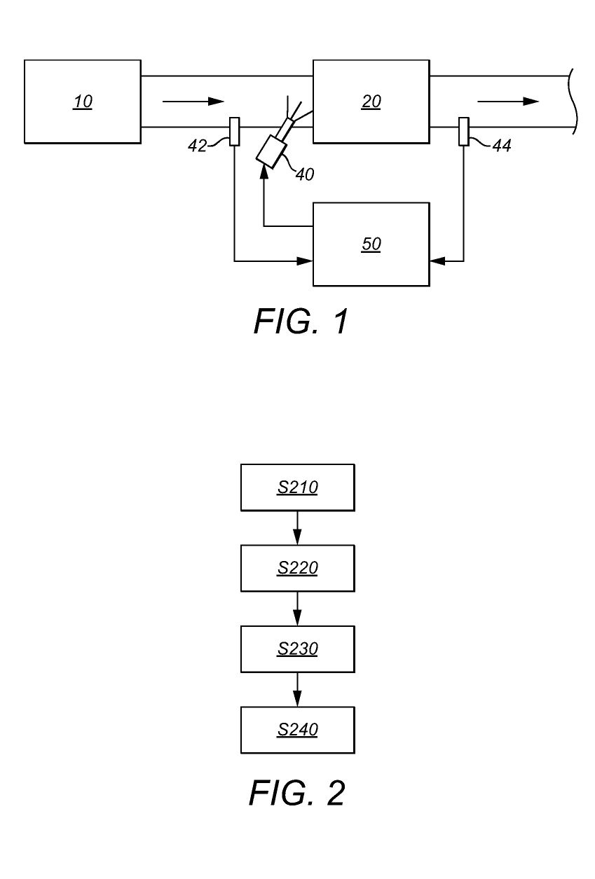

FIG. 2 shows the steps of a method in accordance with a first mode of the present disclosure which may obtain an estimate of the actual NOx output (est(NOxₒᵤₜ)) and ammonia output (est(NH₃)) from the SCR device 20.

In the method shown in FIG. 2, the conversion efficiency based on variance (εᵥₐᵣ) is obtained using the formula above. In order to do this, the variance of NOxᵢₙ and the variance of NOxₒᵤₜ are calculated in step S210 and εᵥₐᵣ is then calculated in step S220.

Having calculated εᵥₐᵣ, a value for est(NOxₒᵤₜ) may be obtained in step S230 as follows:

est(NOxₒᵤₜ)=NOxᵢₙ−ε_{VAR}×NOxᵢₙ=NOxᵢₙ(1−ε_{VAR}) where 0≦ε_{VAR}≦1

An estimate of ammonia slip (est(NH₃)) may then be obtained in step S240 by subtracting est (NOxₒᵤₜ) from the measurement NOxₒᵤₜ from the downstream NOx sensor 44. If only NOx is output from the SCR device 20, est (NOxₒᵤₜ) will be the same as, or very similar to, the measurement NOxₒᵤₜ from the downstream NOx sensor 44. Consequently, est(NH₃) should be 0 or very small.

If ammonia slip is occurring, est(NOxₒᵤₜ) will be less than the measurement NOxₒᵤₜ from the downstream NOx sensor 44. By subtracting est(NOxₒᵤₜ) from NOxₒᵤₜ, an estimate of the ammonia component in NOxₒᵤₜ may be obtained.

It may not be possible for the SCR device 20 to output a negative amount of NOx or ammonia. Therefore, est(NOx) and est(NH3) might be limited to positive values only. That way, if a negative estimate is arrived at, the estimate may be set to 0 so that it may not adversely affect any subsequent calculations or algorithms.

The estimate of ammonia output (est(NH₃)) may then be compared to a threshold value, the outcome of which may determine how the ‘cut dosing flag’ is set. If est(NH₃) is less than or equal to the threshold, it may be assumed that ammonia slip is not occurring and the ‘cut dosing flag’ may be set low. If, however, est(NH₃) is greater than the threshold, it may be assumed that ammonia slip is occurring and the ‘cut dosing flag’ may be set high.

The level of the threshold value may be fixed at a pre-determined value suitable for the internal combustion engine 10 and SCR device 20 in question, for example at a value of 20 ppm.

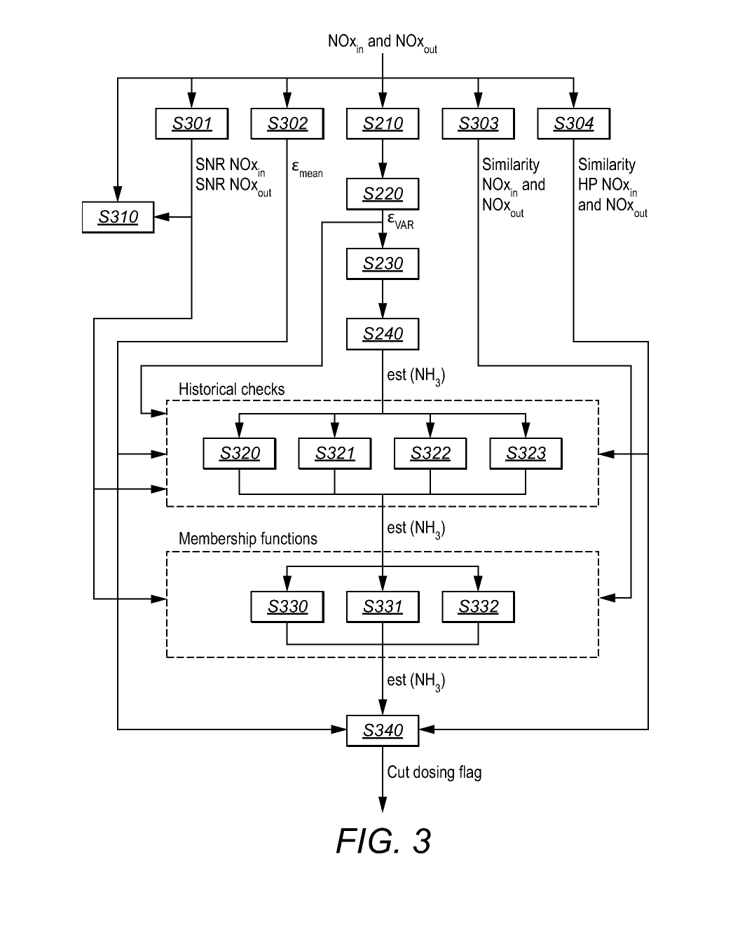

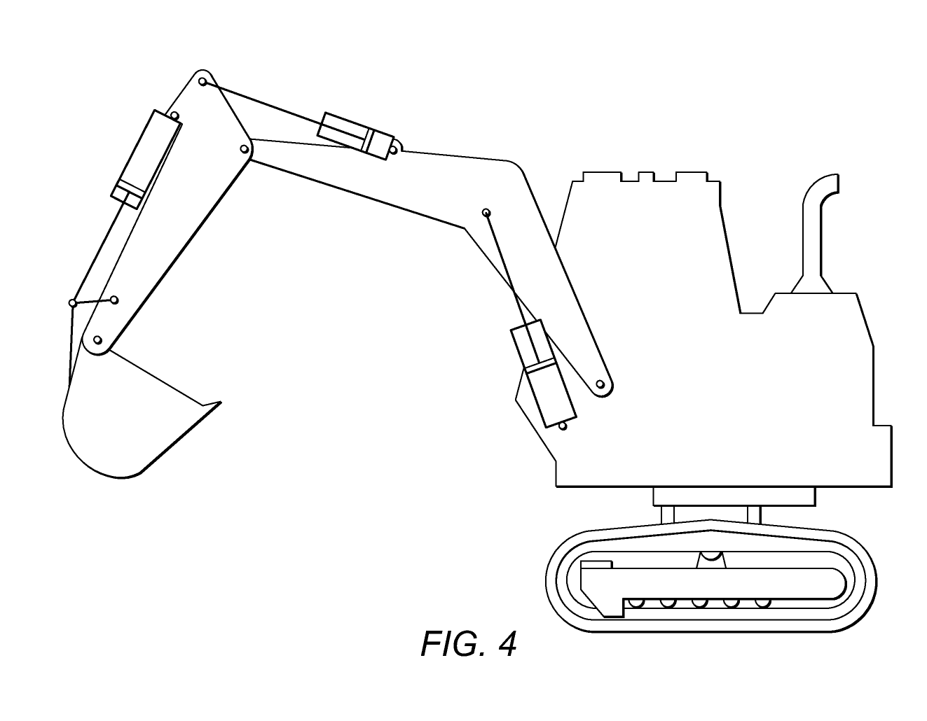

FIG. 3 shows further calculation steps, which might be implemented alongside the method for estimating ammonia output in order to improve the reliability with which the ‘cut dosing flag’ may be set.

Because the calculation of est (NOxₒᵤₜ) and est(NH₃) consider the variance of NOxᵢₙ, it may be useful for the NOxᵢₙ signal to be excited (i.e., not steady state). Engines are usually very transient, which will cause the NOxᵢₙ signal to be excited. However, if the signals are steady, εᵥₐᵣ may not provide an accurate indication of the actual efficiency of the SCR device 20, and therefore est(NOxₒᵤₜ) and est(NH₃) may not be very accurate.

Consequently, it may be useful to monitor how steady or excited the NOxᵢ signal is in order to determine how accurate εᵥₐᵣ may be, and therefore how accurate est(NOxₒᵤₜ) and est(NH₃) might be.

If the NOxₒᵤₜ measurement is quite low, for example below a predetermined threshold set to between 5-100 pm, such as 15 ppm, it may be considered to be of no interest if NOx or ammonia is being output and so the accuracy of est (NOxₒᵤₜ) and est(NH₃) may be irrelevant. In this case, it may not matter if NOxᵢₙ is steady or excited.

However, if the NOxₒᵤₜ measurement is above the predetermined threshold, it may be important to know if it is NOx or ammonia being output so that the dosing can be corrected accordingly. In this case, it is important that NOxᵢₙ is excited so that est (NOxₒᵤₜ) and est(NH₃) may be trusted.

In order to determine if the NOxᵢₙ signal is steady state, the speed of the internal combustion engine 10 might be monitored. If the speed of the internal combustion engine 10 is steady, it may be likely that the NOxᵢₙ signal is steady state. A steady supply of fuel to the internal combustion engine 10 may also be an indicator that NOxᵢₙ may be steady state.

Additionally, or alternatively, a signal to noise ratio (SNR) of NOxᵢₙ may be determined. A high SNR might indicate that NOxᵢₙ is not varying very much (i.e., noise is low) and may therefore indicate that the NOxᵢₙ signal is steady state. SNR of NOxᵢₙ may be determined in step S301 shown in FIG. 3.

It may be arranged that steady state is only considered when NOxₒᵤₜ is above the predetermined threshold and is only determined to be occurring when the engine speed, fuel supply and NOxᵢₙ SNR also all indicate that steady state is occurring. Furthermore, it may be arranged that steady state is only determined to be occurring when the engine speed, fuel supply and NOxᵢₙ SNR have all indicated that steady state is occurring, and when NOxₒᵤₜ has also been above the threshold level, for at least a predetermined period of time.

The SNR of NOxᵢₙ may be determined, for example, using the following formula:

$$SNR = \frac{\overline{{NOx}_{i n}}}{σ} = \frac{\overline{{NOx}_{i n}}}{\sqrt{Cov ( {NOx}_{i n} , {NOx}_{i n} )}}$$

The mean of NOxᵢₙ ( N̅O̅x̅ᵢ̅ₙ̅) may be obtained using a standard moving average (Aₘₒᵥ) over a predetermined time period using the following formula:

$${A}_{mov} = \frac{{x}_{n} + {x}_{n - 1} + … + {x}_{n - k + 1}}{k}$$

However, this technique may require the use of k memory cells. For example, if the time period over which the average is taken is 120 seconds, and the sampling rate of NOxᵢₙ is 1Hz, k=120. This may cause calculation of the mean of NOxᵢₙ using the moving average Aₘₒᵥ technique to be computationally intensive.

Alternatively, an exponentially weighted form of the average may be used, as follows:

$${A}_{EXP} = \frac{{x}_{n} + {px}_{n - 1} + {p}^{2} {x}_{n - 2} + … + {p}^{n - 1} {x}_{n - k + 1}}{1 + p + {p}^{2} + {p}^{3} + … {p}^{n - 1}} where 0 < p < 1$$

By approximating the geometric series denominator to be 1/(1−p),the above formula may be simplified and reduced down to:

A_{EXP}=(1−p)xₙ+pA_{EXP(n-1)}

The difference equation may be represented using z transform theory as:

$$\frac{{A}_{EXP}}{x} = \frac{z ( 1 - p )}{z - p}$$

And by replacing K=1−p, you may arrive at:

$$\frac{{A}_{EXP}}{x} = \frac{Kz}{z + ( K - 1 )}$$

Thus, the exponentially weighted average A_{EXP} is a first order difference equation, such that where K=131 e^{2nfT}, calculation of the exponentially weighted average A_{ERP} may be implemented using a single pole, low pass filter.

As can be seen from the presence of only a single ‘x’ and one previous A_{EXP}(n−1) term (or the single delay operator z in the z transform theory equations above), by using a single pole, low pass filter to calculate an exponentially weighted average of NOxᵢₙ, only instantaneous measurements of NOxᵢₙ from the upstream NOx sensor 42 and one previous A_{EXP} output are required. Thus, A_{EXP} may be calculated using only a single memory cell to store the old output value, which therefore requires less memory space, and is less computationally intensive, than calculating the mean NOxᵢₙ using A_{EXP}.

The covariance term (cov(NOxᵢₙ, NOxᵢₙ)) in the SNR formula above may be determined using the usual definition of covariance:

Cov(x, y)=E[(x−E[x])(y−E[y])]

wherein, the expectation operators E are the same as the moving average Aₘₒᵥ, above, but may readily be approximated for ease of calculation using A_{EXP}.

When calculating the covariance, the above formula may be arranged to:

Cov(x, y)=E[xy]−E[x]E[y]

Therefore, Cov(NOxᵢₙ, NOxᵢₙ) may be calculated by setting x=y=NOxᵢₙ and determining the expectation operators E, i.e., N̅O̅x̅ᵢ̅ₙ̅, using the A_{EXP} technique described earlier.

This SNR NOxᵢₙ value may then be used in the steady state determination step, S310, along with a measure of the internal combustion engine 10 speed, fuel quantity and NOxₒᵤₜ.

If it is determined that the NOxᵢₙ signal is steady state because the SNR NOxᵢₙ, engine speed and fuel quantity have all indicated this for a sufficient period of time, actions may be taken in order to excite the NOxᵢₙ signal and thereby improve the accuracy of εᵥₐᵣ and consequently est (NOxₒᵤₜ) and est(NH₃).

The NOxᵢₙ signal may be artificially excited by alternating the CO₂:O₂ ratio in the inlet manifold of the internal combustion engine 10. If the internal combustion engine 10 has an Exhaust Gas Recirculation system (EGR), the CO₂:O₂ ratio may be alternated, for example, by applying an alternating signal to the signal that controls the EGR valve. If the internal combustion engine 10 is turbocharged, the CO₂:O₂ ratio may be alternated, for example, by applying an alternating signal to the signal that controls the wastegate or variable-geometry turbine (VGT) of the turbocharger.

The shape, period and amplitude of the alternating signal may be varied and increased over time in order more strongly to excite NOxᵢₙ.

Once artificial excitation has begun, est(NOxₒᵤₜ) and est(NH₃) may be trusted once again and used to control dosing of the SCR device 20. Artificial excitation may continue until the NOxₒᵤₜ signal falls back below the threshold (e.g. 20 ppm), which may indicate that it is irrelevant if NOx or ammonia is being sensed, or until the engine speed and fuel quantity change to indicate non-steady state conditions, at which time artificial excitation may no longer be required. Otherwise, artificial excitation may continue until the expiry of a pre-determined time period, for example 5 minutes.

The frequency response of the est(NH₃) figure might be improved by determining est(NOxₒᵤₜ) using the average of NOxᵢₙ:

est( N̅O̅x̅ₒ̅ᵤ̅ₜ̅)= N̅O̅x̅ᵢ̅ₙ̅−ε_{VAR}× N̅O̅x̅ᵢ̅ₙ̅= N̅O̅x̅ᵢ̅ₙ̅(1−ε_{VAR}) where 0 ≦ε_{VAR}>1

The average of NOxᵢₙ may be determined using the moving average, Aₘₒᵥ, or the weighted exponential average A_{EXP}, techniques described earlier.

If the A_{ERP} technique is used to determine N̅O̅x̅ᵢ̅ₙ̅, N̅O̅x̅ᵢ̅ₙ̅ will also be low pass filtered. As previously explained, the ammonia part of the NOxₒᵤₜ signal read from the downstream NOx sensor is a low frequency component. By low-pass filtering NOxᵢₙ in order to calculate est( N̅O̅x̅ₒ̅ᵤ̅ₜ̅), as above, the estimated NOxₒᵤₜ signal may already be in the correct frequency range for determining an estimate of ammonia out. Furthermore, because A_{ERP} weights the most recent measurements of NOxₒᵤₜ more heavily than older measurements, the function progressively ‘forgets’ about older measurements, which might become increasingly less relevant to the current operation of the SCR device 20.

The estimate of average ammonia out (est( N̅H̅₃̅)) might be determined in an analogous manner to that explained above in respect of est (NOxₒᵤₜ) : by subtracting est( N̅O̅x̅ₒ̅ᵤ̅ₜ̅) from N̅O̅x̅ₒ̅ᵤ̅ₜ̅.

Determining est( N̅H̅₃̅) rather than est(NH₃) might also provide a longer term, rather than instantaneous, indication of ammonia output levels.

There are a number of factors that might reduce the accuracy of the estimates of NOx output (est(NOxₒᵤₜ) or est( N̅O̅x̅ₒ̅ᵤ̅ₜ̅)) and consequently the estimates of ammonia output (est(NH₃) or est( N̅H̅₃̅)). These might include the transport delay of gases through the SCR device 20 and sensor noise and error from the upstream 42 and downstream 44 NOx sensors and other dynamic uncertainties.

Consequently, a number of further checks and calculations might be implemented.

For example, the signal to noise ratio (SNR) of the NOxₒᵤₜ measurement from the downstream NOx sensor 44 might be determined in Step S301 using the same technique described above in respect of NOxᵢₙ. A high SNR of NOxₒᵤₜ suggests that ammonia slip may be likely. This is because, as explained above, the ammonia component of the NOxₒᵤₜ signal is a low frequency component. A high SNR suggests a signal that is relatively high compared with the signal noise. Since signal noise is dominated by high-frequency noise, a high SNR suggests that there is very little noise, i.e., very little NOx, in the NOxₒᵤₜ signal. This means that it may be likely that there is ammonia slip, as long as the NOxᵢₙ signal is excited at the time. When SNR NOxₒᵤₜ is low, it may be unlikely that there is ammonia slip.

The SNR NOxₒᵤₜ may be used by a ‘membership function’ in Step S330 that may modify the magnitude of the ammonia estimate (est (NH₃) or est( N̅H̅₃̅)) . If a high NOxₒᵤₜ SNR is determined, est(NH₃) or est( N̅H̅₃̅) may remain unmodified. However, as NOxₒᵤₜ SNR decreases, the magnitude of est (NH₃) or est( N̅H̅₃̅) may be modified by multiplying it by a fraction between 0 and 1. This fraction may decrease as NOxₒᵤₜ SNR decreases. For example, for a very low NOxₒᵤₜ SNR, the fraction may be small or even 0, such that est (NH₃) or est( N̅H̅₃̅) may become 0, and for a very high NOxₒᵤₜ SNR, the fraction may be large or even 1, such that est (NH₃) or est( N̅H̅₃̅) may remain largely unmodified . The multiplying fraction may be determined using a look-up table with the NOxₒᵤₜ SNR as an input.

A similar multiplying fraction may be determined by considering NOxᵢₙ SNR in a further membership function in Step S331. As already explained, when NOxᵢₙ SNR is high, NOxᵢₙ may be steady state and est (NOxₒᵤₜ) est( N̅O̅x̅ₒ̅ᵤ̅ₜ̅) , est (NH₃) and est( N̅H̅₃̅) may not be accurate. Therefore, this membership function may multiply the estimate of ammonia output by a fraction between 0 and 1, which reduces as NOxᵢₙ SNR increases. For a very high NOxᵢₙ SNR the fraction may be very low or even 0 and for a very low NOxᵢₙ SNR the fraction may be very high or even 1. The multiplying fraction may again be determined using a look-up table with the NOxᵢₙ SNR as an input, and may be calibrated such that the multiplying fraction is equal to 0 at the level of NOxᵢₙ SNR at which steady state conditions are deemed to be occurring and artificial excitation techniques may implemented, as explained earlier. As soon as the artificial excitation techniques are implemented, NOxᵢₙ SNR should decrease so that the estimate of ammonia output may be less affected by this membership function.

A further membership function may apply a multiplying fraction determined by considering the similarly between the NOxᵢₙ and NOxₒᵤₜ signals in Step S332.

The similarity between these two signals may be determined using any technique well known to the skilled person, for example, using the Pearson Product Moment Correlation Coefficient, ρ(x,y). This may be determined using the formula:

$$ρ ( x , y ) = \frac{Cov ( x , y )}{{σ}_{x} {σ}_{y}} = \frac{E [ xy ] - E [ x ] E [ y ]}{\sqrt{Cov ( x , x ) Cov ( y , y )}}$$

The expectation operators E are the same as the moving average Aₘₒᵥ above, but may readily be approximated for ease of calculation using A_{EXP}. The covariance functions may be calculated using the techniques described earlier in respect of SNR calculations. The similarity of NOxᵢₙ and NOxₒᵤₜ may be determined by substituting x=NOx_{in·}and y=NOx_{out.}

If there is perfect positive correlation the function may return 1; perfect negative correlation the function may return −1; and no correlation the function may return 0. A value of, for example, 0.5 might indicate that there is some positive correlation between the figures, but that there are some residual errors.

If there is a high degree of correlation between the NOxᵢₙ and NOxₒᵤₜ signals, the estimate of ammonia out may be untrustworthy and may be reduced or driven to zero by the multiplying fraction. If there is a low degree correlation, the estimate of ammonia out may be increasingly trustworthy as the correlation value decreases towards zero, so the multiplication fraction may be increased as the correlation value decreases towards zero. If there is negative correlation, it may also be assumed that estimates of ammonia out are trustworthy. The multiplying fraction may be determined using a look-up table with the correlation figure as an input.

If two or more of the above membership functions are used, their inputs may form the inputs to a look-up table that may determine a single multiplying fraction to be applied to the estimated ammonia output (est (NH₃) or est( N̅H̅₃̅)) before the estimate is passed on to any subsequent functions or calculations.

Other checks, referred to herein as historical checks, may also be implemented before or after the membership functions in order to determine whether the estimate is trustworthy enough to be allowed to proceed on to any further functions. It may be that only a single historical check is implemented on its own, or that two or more of the historical checks are implemented in parallel. Where multiple historical checks are implemented in parallel, it may be arranged such that only one of the checks needs to determine that ammonia slip is likely for the ammonia output estimate to be passed on to the next function.

Furthermore, if at least one of the historical checks determines that NOx output is likely, the ammonia output estimate may be blocked by, for example, setting the ammonia output estimate to 0. In this arrangement, where there is a conflict and one historical check believes that ammonia slip is likely and another believes that NOx output is likely, it may be arranged that the ammonia output estimate is blocked in order to maintain SCR device 20 dosing to keep as a priority the reduction of NOx emissions into the atmosphere.

One historical check might consider the mean conversion efficiency (εₘₑₐₙ) alongside the similarity of the high-frequency components of NOxᵢₙ and NOxₒᵤₜ signal, obtained by high-pass filtered (HP) NOxᵢₙ and NOxₒᵤₜ signals, which is carried out in Step S320. If it is also determined that εₘₑₐₙ has already reached a peak value (i.e., εₘₑₐₙ reached a peak value and has started to decrease), it may be considered that ammonia slip is possible. If it is considered that HP NOxᵢₙ and NOxₒᵤₜ signals have also lost similarity, it may then be determined that ammonia slip is likely. If both the mean conversion efficiency εₘₑₐₙ peak value check and the HP NOxᵢₙ and NOxₒᵤₜ similarity indicate that ammonia slip is likely, the historical check may ensure that the estimate of ammonia output be passed to the next function, for example the membership functions described above, or the function to compare the estimated ammonia output with a threshold value in order to set the ‘cut dosing flag’.

If, however, it is determined that εₘₑₐₙ has not yet reached a peak value, it may be considered that NOx output is possible. If it is considered that HP NOxᵢₙ and NOxₒᵤₜ signals also have a high degree of similarity, it may then be determined that NOx output is likely. If both the mean conversion efficiency εₘₑₐₙ peak value check and the HP NOxᵢₙ and NOxₒᵤₜ similarity indicate that NOx output is likely, the historical check may ensure that the estimate of ammonia output is blocked, for example by setting it to 0, such that an ammonia estimate of zero is passed on to the next function, for example the membership functions described above, or the function to compare the estimated ammonia output with a threshold value in order to set the ‘cut dosing flag’.

The NOxᵢₙ and NOxₒᵤₜ may be high pass filtered using any standard technique known to the skilled person. For example, it may be obtained by subtracting a low pass filtered signal from the original signal. The low pass filtered signal might be obtained using the A_{EXP} function explained earlier. In this instance, for example, HP NOxᵢₙ=NOxᵢₙ− N̅O̅x̅ᵢ̅ₙ̅, wherein N̅O̅x̅ᵢ̅ₙ̅, is calculated using the A_{EXP} function.

Similarity between HP NOxᵢₙ and NOxₒᵤₜ may be determined using any technique well known to the skilled person, for example, using the Pearson Product Moment Correlation Coefficient p(x,y) explained earlier. Good similarity may indicate that the downstream NOx sensor 44 is measuring only NOx gases, in which case the ammonia estimate signal is likely to be untrustworthy and should not be allowed to continue. On the other hand, poor similarity may indicate that the downstream NOx sensor 44 is detecting ammonia slip and so the historical check should ensure the ammonia output estimate be passed on to the next function. The threshold of similarity value below which this historical check determines that ammonia slip might be occurring and the threshold above which this historical check determines that NOx output might be occurring may be set to be any suitable value between 0 and 1. For example, a similarity value of 0.8 or more may be set to indicate that NOx output might be occurring, and a similarity value of 0.2 or less may be set to indicate that ammonia output might be occurring.

Where the similarity value is between the threshold where ammonia-slip might be occurring and the threshold where NOx output might be occurring, for example a similarity value of 0.5, the historical checks may be configured to allow the current blocking or passing of ammonia output estimate to continue. For example, if the similarity value had been very high such that that ammonia output estimate is blocked, this blocking may be allowed to continue until a historical check determines that ammonia-slip is likely, for example the similarity value is below the ammonia output possible threshold (which is, in this example, 0.2 or less) and the mean conversion efficiency εₘₑₐₙ indicates that ammonia-slip is possible. Likewise, where the similarity value had been very low such that the ammonia output estimate is allowed to pass, the ammonia output may continue to be allowed to pass until a historical check determines that NOx output is likely, for example when the similarity value exceeds the NOx output possible threshold (which is, in this example, 0.8 or more) and the mean conversion efficiency εₘₑₐₙ indicates that NOx output is possible.

Another historical check, carried out in Step S321, might be to consider the level of the estimated ammonia output (est(NH3) or est( N̅H̅3̅)) and the similarity between the high-pass filtered (HP) NOxᵢₙ and NOxₒᵤₜ signals. If both the estimated ammonia output exceeds a pre-determined threshold value and the similarity figure is below a pre-determined threshold value, it may be deemed that ammonia slip is likely and the historical check may ensure that the estimate of ammonia output be allowed to pass to the next function. However, if the estimated ammonia output is less than a pre-determined threshold value and the similarity figure is above a pre-determined threshold value, it may be deemed that NOx output is likely and the historical check may block the estimated ammonia output, for example by setting it to zero. The pre-determined threshold values may be set by the skilled person at any suitable level given the internal combustion engine 10 and SCR device 20.

This historical check could be implemented by a plurality of similar checks running in parallel with each other. Each of the plurality of checks might have different threshold levels—for example, one check might determine that ammonia—slip is likely if the ammonia estimate exceeds a relatively high ammonia output estimate threshold, such as 90ppm, and the similarity figure is below a relatively high cross—correlation threshold, such as 0.4, and another check might determine that ammonia—slip is likely if the ammonia estimate exceeds a relatively low ammonia output estimate threshold, such as 60 ppm, and the similarity figure is below a relatively low cross-correlation threshold, such as 0.1. Similar parallel checks may be arranged to determine if NOx output is likely, wherein if at least one ammonia output likely check determines that ammonia output is likely, the ammonia estimate may be allowed to pass to the next function, and if at least one NOx output likely check determines that NOx output is likely, the ammonia estimate may be blocked, for example by setting it to zero.

Another historical check, carried out in Step S322, might be to compare εₘₑₐₙ and εᵥₐᵣ. As explained earlier, when ammonia slip is taking place, εₘₑₐₙ may be less than εᵥₐᵣ. Therefore, if εₘₑₐₙ is well below εᵥₐᵣ, εᵥₐᵣ ammonia slip may be highly likely and the historical check should ensure that the estimate of ammonia output be allowed to pass to the next function. The amount by which εₘₑₐₙ may be below εᵥₐᵣ in this check before ammonia slip is deemed to be likely, may be predetermined by the skilled person on the basis of the internal combustion engine 10 and SCR device 20 in use.

Another historical check, carried out in Step S323, might be to consider the mean conversion efficiency (εₘₑₐₙ) If εₘₑₐₙ is negative, NOxₒᵤₜ must be greater than NOxᵢₙ, which means that ammonia slip may be likely. Therefore, if εₘₑₐₙ<0, the historical check may ensure that the estimate of ammonia slip is allowed to pass to the next function.

As explained above, if any one of the historical checks determines that ammonia output is likely, it may be arranged that the ammonia estimate is allowed to pass to the next function. However, if any one of the historical checks that considers if NOx output is likely determines that NOx output is likely, it may be arranged that the ammonia estimate is blocked, for example by setting it to zero. If there is a disagreement and one historical check considers ammonia output to be likely and another considers NOx output to be likely, it may be arranged that the ammonia estimate is blocked, for example by setting it to zero, in order to allow SCR device 20 dosing to continue so as to prioritise NOx output emission reduction.

The usefulness of the high-pass filtered (HP) NOxᵢₙ and HP NOxₒᵤₜ signal similarity value determined in the historical checks above may be improved by utilising a time delay to overcome inaccuracies caused by the ‘transport delay’ of the SCR device 20.

The ‘transport delay’ is the time it may take exhaust gasses to pass through the SCR device 20. A volume of gas that is sensed by the upstream NOx sensor 42 may take some time, for example 2 seconds, to travel through the SCR device 20 and be sensed by the downstream NOx sensor 44. This time delay may be referred to as the ‘transport delay’. Consequently, readings taken from the upstream NOx sensor 42 and the downstream NOx sensor 44 at the same time may not correspond to the same volume of gas, since that volume of gas may take an amount of time equal to the transport delay to pass through the SCR device 20. This may result in inaccuracies in the estimated NOx and ammonia outputs.

Multiple similarity values may be calculated, each with a different time delay applied between the NOx_{in·}and NOxₒᵤₜ signal. For example, if a time delay of 1 second is used, the NOxᵢₙ signal used for the HP similarity calculation will be the signal obtained one second before the time at which the NOxₒᵤₜ signal used for the HP filtered similarity calculation is obtained. The maximum similarity value obtained out of all of the different time delays may indicate the time delay that most closely matches the transport delay of the SCR device 20. This is because the NOxᵢₙ and NOxₒᵤₜ values at that time delay should be most closely aligned and therefore obtain a higher similarity value than when the NOxᵢₙ and NOxₒᵤₜ signals are misaligned by a large difference between the time delay and the transport delay.

If this transport delay optimisation technique is used, the maximum HP NOxᵢₙ and NOxₒᵤₜ similarity value may be the value that is used in the historical checks above. Furthermore, the time delay value determined by this technique may be used by other functions that compare NOxᵢₙ and NOxₒᵤₜ signals, for example efficiency (εₘₑₐₙ and ε_{VAR}) calculations in Steps S220 and S302. Alternatively, a fixed time delay may be used by all of the relevant functions.

In addition to, or as an alternative to, setting the ‘cut dosing flag’ by comparing the estimated ammonia output to a predetermined threshold level, other factors might be used to set the flag in Step S340.

If εₘₑₐₙ is negative, which may be caused by NOxₒᵤₜ being greater than NOxᵢₙ, it may be considered that ammonia slip must be taking place. Therefore, εₘₑₐₙ may be monitored and if it goes below zero, the ‘cut dosing flag’ may be set and may only be allowed to reset after εₘₑₐₙ becomes positive again.

Furthermore, if εₘₑₐₙ has a very high value, for example above 0.97, it may be considered that ammonia slip may occur in the near future and a lower conversion efficiency may be tolerated in order to ensure that ammonia slip does not occur in the near future. Therefore, the ‘cut dosing flag’ may be set as a preventative measure, and only reset to allow dosing after εₘₑₐₙ has gone back below the threshold value of, for example, 0.97.

It may be arranged that if two or more of these tests for setting the ‘cut dosing flag’ are set up in parallel, the criteria of only one of the test needs to be met in order to set the ‘cutting dosing flag’ (i.e., the ammonia estimate goes above the threshold, or εₘₑₐₙ, goes negative or εₘₑₐₙ is very high).

If the ‘cut dosing flag’ is set to stop dosing, it may be reset to allow dosing when it is determined that ammonia slip is no longer taking place.

This may be determined when the signal similarity of HP NOxᵢₙ and HP NOxₒᵤₜ is high, as this may indicate that the downstream NOx sensor 44 is measuring only NOx and that ammonia is longer present.

FIG. 1 shows a controller 30 in accordance with an embodiment of the present disclosure.

The controller 50 may be configured to carry out the method steps described in the present disclosure.

The controller 50 may have a number of inputs and outputs that may be used in order to determine an estimate of ammonia output from the SCR device 20 and in order to set the ‘cut dosing flag’ that might be used to control the injector 40. For example, the inputs might include, but are not limited to, a NOxᵢₙ reading from the upstream NOx sensor 42 and a NOxₒᵤₜ reading from the downstream NOx sensor 44. The controller 50 may also have a number of outputs, including, but not exclusive to, a control signal for the injector 40.

The controller 30 may be implemented in an engine control unit, for example the Caterpillar® A4:E4 or A5:E2, or as a standalone control unit.

FIG. 1 also shows an SCR system comprising an SCR device 20 and the controller 50, which may be arranged to determine the ammonia output of the SCR device 20 and to control the injector 40. Furthermore, FIG. 1 also shows an engine unit comprising an internal combustion engine 10 and the SCR system.

FIG. 4 shows a vehicle within which the engine unit shown in FIG. 1 could be used.

### INDUSTRIAL APPLICABILITY

The present disclosure finds application in determining an estimate of ammonia output from an SCR device. In order to estimate the ammonia output, a NOx conversion efficiency of the SCR device is determined from a variance of a NOx input measurement and a variance of a NOx output measurement. An estimate of NOx output is then determined using the NOx conversion efficiency and the NOx input measurement and an estimate of ammonia output may then be found from the estimate of NOx output and the NOx output measurement.

By determining the NOx coversion efficiency from variance values, the cross-sensitivity of the NOx output sensor to NOx and ammonia may be overcome and a more accurate estimate of ammonia output may be determined. When the NOx output sensor is measuring ammonia, the ammonia component of the NOx output sensor measurement is low frequency and therefore has little or no effect on the variance of the NOx output measurement. Therefore, considering the variance of the NOx output measurement when calculating the conversion efficiency may eliminate any ammonia measurements from the NOx output measurement used for conversion efficiency so that a more accurate estimate of ammonia output may be determined using a NOx input sensor and a NOx output sensor. A more accurate measure of ammonia output may enable SCR device dosing to be cut with more accuracy when ammonia slip is taking place, which may result in better dosing of the SCR device and therefore less NOx and ammonia output from the SCR device.

It may be arranged that an estimated average ammonia output is determined to provide a longer term estimate of ammonia output, which may be more reliable for identifying dosing errors and therefore controlling dosing of the SCR device.

It might also be arranged that the estimate of ammonia output is passed through a series of ‘historical checks’ andor ‘membership functions’ in order to improve the accuracy and reliability of the estimate of ammonia output. In this way, doubtful ammonia output estimates may be scaled down or set to zero in order to limit or prevent their impact on the control of the SCR device.

Furthermore, it may be arranged that a time delay is applied in respect of the measured NOx input and the measured NOx output to overcome the ‘transport delay’ of the SCR device.

Multiple different time delays may be applied in parallel and a similarity value for a high pass filtered measured NOx input and a high pass filtered measured NOx output may be calculated for each time delay. The time delay that results in the best similarity between the high pass filtered measured NOx input and the high pass filtered measured NOx output may represent the time delay that most accurately corresponds to the transport delay and may then be used when calculating an estimate of ammonia output. In this way, the accuracy with which the transport delay of the SCR device is compensated for may be improved and changes in the transport delay of the SCR device may be identified and compensated for with more accuracy, thereby improving the accuracy of the estimate of ammonia output.

1. A method for estimating an amount of ammonia output from a selective catalytic reduction (SCR) device, the method comprising the steps of:
determining a NOx conversion efficiency of the SCR device from a variance of a NOx sensor measurement signal at an input of the SCR device and a variance of a NOx sensor measurement signal at an output of the SCR device;
estimating an amount of NOx output from the SCR device from the NOx conversion efficiency of the SCR device and a NOx sensor measurement signal at an input to the SCR device; and
estimating the amount of ammonia output from the SCR device from the estimated NOx output and the NOx sensor measurement signal at the output of the SCR device.

2. The method of claim 1, wherein the estimated amount of NOx output from the SCR device is an estimated average NOx output, which is estimated using the NOx conversion efficiency and an average of the NOx sensor measurement signal at the input of the SCR device; and
the estimated amount of ammonia output from the SCR device is an estimated average ammonia output, which is estimated from the estimated average NOx output and an average NOx sensor measurement signal at the output of the SCR device.

3. The method of claim 2, wherein the average NOx sensor measurement signal at the input of the SCR device is determined using a low pass filter and the average NOx sensor measurement signal at the output of the SCR device is determined using a low pass filter.

4. The method of claim 1, wherein the estimated ammonia output or estimated average ammonia output is adjusted in magnitude on the basis of a signal to noise ratio of the NOx sensor measurement signal at the input to the SCR device.

5. The method of claim 1, wherein the estimated ammonia output or estimated average ammonia output is adjusted in magnitude on the basis of a signal to noise ratio of the NOx sensor measurement signal at the output of the SCR device.

6. The method of claim 1, wherein the estimated ammonia output or estimated average ammonia output is adjusted in magnitude on the basis of a similarity of the NOx sensor measurement signal at the input of the SCR device and the NOx sensor measurement signal at the output of the SCR device.

7. The method of claim 1, further comprising the steps of:
determining an average NOx conversion efficiency of the SCR device from the average of the NOx sensor measurement signal at the input of the SCR device and the average of the NOx sensor measurement signal at the output of the SCR device; and
setting the estimated ammonia output or estimated average ammonia output to zero if the average NOx conversion efficiency and a similarity of high-frequency components of the NOx sensor measurement signal at the output of the SCR device and high-frequency components of the NOx sensor measurement signal at the input of the SCR device indicate that ammonia slip from the SCR device is unlikely.

8. The method of claim 1, further comprising the steps of:
determining the average NOx conversion efficiency of the SCR device from the average of the NOx sensor measurement signal at the input of the SCR device and the average of the NOx sensor measurement signal at the output of the SCR device; and
setting the estimated ammonia output or estimated average ammonia output to zero if a comparison of the average NOx conversion efficiency and the NOx conversion efficiency determined using variance indicates that ammonia slip from the SCR device is unlikely.

9. The method of claim 1, further comprising the steps of:
determining the average NOx conversion efficiency of the SCR device from the average of the NOx sensor measurement signal at the input of the SCR device and the average of the NOx sensor measurement signal at the output of the SCR device; and
setting the estimated ammonia output or estimated average ammonia output to zero if the average NOx conversion efficiency is below zero.

10. The method of claims claim 1, wherein the estimated ammonia output or estimated average ammonia output is set to zero if the estimated ammonia output or estimated average ammonia output and the similarity of the high-frequency components of the NOx sensor measurement signal at the output of the SCR device and the high-frequency components of the NOx sensor measurement signal at the output of the SCR device indicate that ammonia slip from the SCR device is unlikely.

11. The method of claim 1, wherein dosing of the SCR device is stopped if the estimated ammonia output or estimated average ammonia output exceed a threshold value.

12. A controller for estimating an amount of ammonia output from a selective catalytic reduction (SCR) device, the controller being configured to:
determine a NOx conversion efficiency of the SCR device from a variance of a NOx sensor measurement signal at an input of the SCR device and a variance of a NOx sensor measurement signal at an output of the SCR device;
estimate an amount of NOx output from the SCR device from the NOx conversion efficiency of the SCR device and a NOx sensor measurement signal at the input to the SCR device; and
estimate an amount of ammonia output from the SCR device from an estimated NOx output and the NOx sensor measurement signal at the output of the SCR device.

13. An SCR system comprising:
an SCR device, and p1 the controller defined in claim 12, the controller being arranged to determine the amount of ammonia output from the SCR device.

14. An internal combustion engine comprising the SCR system defined in claim 13.

15. A vehicle comprising the internal combustion engine defined in claim 14.

16. The method of claim 2, wherein the estimated ammonia output or estimated average ammonia output is adjusted in magnitude on the basis of a signal to noise ratio of the NOx sensor measurement signal at the input to the SCR device.

17. The method of claim 2, wherein the estimated ammonia output or estimated average ammonia output is adjusted in magnitude on the basis of a signal to noise ratio of the NOx sensor measurement signal at the output of the SCR device.

18. The method of claim 2, wherein the estimated ammonia output or estimated average ammonia output is adjusted in magnitude on the basis of a similarity of the NOx sensor measurement signal at the input of the SCR device and the NOx sensor measurement signal at the output of the SCR device.

19. The method of claim 2, further comprising the steps of:
determining an average NOx conversion efficiency of the SCR device from the average of the NOx sensor measurement signal at the input of the SCR device and the average of the NOx sensor measurement signal at the output of the SCR device; and
setting the estimated ammonia output or estimated average ammonia output to zero if the average NOx conversion efficiency and a similarity of high-frequency components of the NOx sensor measurement signal at the output of the SCR device and high-frequency components of the NOx sensor measurement signal at the input of the SCR device indicate that ammonia slip from the SCR device is unlikely.

20. The method of claim 2, further comprising the steps of:
determining the average NOx conversion efficiency of the SCR device from the average of the NOx sensor measurement signal at the input of the SCR device and the average of the NOx sensor measurement signal at the output of the SCR device; and
setting the estimated ammonia output or estimated average ammonia output to zero if a comparison of the average NOx conversion efficiency and the NOx conversion efficiency determined using variance indicates that ammonia slip from the SCR device is unlikely.
